(19) United States
(12) Patent Application Publication
(10) Pub. No.: US 2019/0228850 A1
(43) Pub. Date: Jul. 25, 2019

(54) INTERACTIVE PILL DISPENSING APPARATUS AND ECOSYSTEM FOR MEDICATION MANAGEMENT

(71) Applicant: Elements of Genius, Inc., Mt. Pleasant, SC (US)

(72) Inventor: Jonathan E. Ramaci, Mt. Pleasant, SC (US)

(21) Appl. No.: 16/257,875

(22) Filed: Jan. 25, 2019

Related U.S. Application Data
(60) Provisional application No. 62/621,803, filed on Jan. 25, 2018.

(51) Int. Cl. G16H 20/13; A61J 7/04; G16H 50/20; G16H 80/00

(52) U.S. Cl. CPC G16H 20/13; A61J 7/0481; G16H 40/63; G16H 80/00; A61J 2200/30; G16H 50/20

(57) ABSTRACT

An interactive pill dispensing system, apparatus, and methods with advanced capabilities for medication management and adherence. In the broadest terms, the system comprises an advanced pill dispensing apparatus with one or more user-interfaces, computing, mobile communication, networking, and Artificial Intelligent (AI) digital assistant capabilities. These capabilities enable the pill dispensing user (patient) to be connected to a support system of care givers. The apparatus functions as an integral component of an ecosystem for medication management and adherence. The ecosystem comprises one or more said apparatus, local area network (LAN), wide area network (WAN), Internet of Things (IoTs), a cellular network, Internet, ISP, mobile cellular phone, local server, remote server, cloud server, cloud computing services, laptop, desktop computer, database, application software, smart appliances. One or more of said components are configured to enable seamless communication between patient, caregivers, healthcare providers, and service providers.

### CROSS-REFERENCE TO RELATED APPLICATIONS

This application claims the benefit of U.S. Provisional Application 62/621,803, entitled “INTERACTIVE PILL DISPENSING APPARATUS AND ECOSYSTEM FOR MEDICATION MANAGEMENT,” filed Jan. 25, 2018 and hereby incorporated by reference in its entirety.

### FIELD

The present disclosure relates to the field of dispensing oral medication. In particular, the disclosure relates to a system, apparatus, and methods for medication management and adherence incorporating a voice-controlled relational agent or AI digital assistant.

BACKGROUND

Adherence to long-term medical therapy is the extent to which a person's behavior (e.g., taking medication, following a diet, masking lifestyle changes) is aligned with and in conformance with recommendations from their healthcare provider. Nonadherence occurs when a patient fails to use the prescribed treatment correctly, such as taking an incorrect amount of medication or at the wrong time. Nonadherence is estimated at a rate of 50% often leading to avoidable hospitalization, and has a significant financial impact on healthcare systems, nationally and worldwide.

Medication nonadherence is multifactorial and possible causes include cognitive function, psychological issues, self-reliance, memory-issues, health literacy, polypharmacy, and the lack of a care support system. There are two types of nonadherence: unintentional and intentional, unintentional nonadherence occurring when patients involuntarily fail to use the prescribed treatment. These factors and behaviors may put patients at the highest risk for medication nonadherence. Conventional interventions for patients demonstrating tendencies towards medication nonadherence incorporate one or methods such as counseling, telephone calls, message alerts and use of pillboxes. However, it is challenging to implement these methods in routine healthcare. The proliferation of computing and networking technologies and smaller form factor computing devices such as tablets, smart phones, etc. are increasingly used for personal purposes. These devices, for example smart phones, have been proposed to provide communication and connectivity to stand-alone automated pill dispensers, enabling care givers or healthcare providers to provide more effective interventions to patients. Despite patients' increasing use of these more sophisticated devices (e.g. with communication and connectivity functions) to manage their daily activities and healthcare routines, improvements on existing pill dispensing apparatuses and medication management methods are necessary to optimize patient compliance and medication adherence.

### SUMMARY

Disclosed are an interactive pill dispensing system, apparatus, and methods with advanced capabilities for medication management and adherence. In the broadest terms, the system comprises an advanced pill dispensing apparatus with one or more user-interfaces (e.g., voice-controlled), computing, mobile communication, networking, and Artificial Intelligent (AI) digital assistant capabilities. These capabilities enable the pill dispensing user (patient) to be connected to a support system of care givers (e.g., family member, caregiver, etc., healthcare providers (e.g., nurse, physicians), and service providers (e.g., pharmacist, wholesalers, pharmacy benefit manager (PBM)). The apparatus functions as an integral component of an ecosystem for medication management and adherence. The ecosystem comprises one or more said apparatus, local area network (LAN), wide area network (WAN), Internet of Things (IoTs), a cellular network, Internet, ISP, mobile cellular phone (e.g., smart phone), local server, remote server, cloud server, cloud computing services, laptop, desktop computer, database, application software, smart appliances (e.g., Amazon Echo®/Dot®, TV, etc.). One or more of said components are configured to enable seamless communication between patient, caregivers, healthcare providers, and service providers.

According to an aspect of the present disclosure, an interactive pill dispensing apparatus provides one or more user functions including, but not limited to, voice, data, SMS reminders, alerts, medication adherence monitoring, location via SMS, GPS location, and 911 emergency services. The said device incorporates, within or user accessible of a self-contained portable unit, one or more microprocessor, microcontroller, micro GSM/GPRS chipset, micro SIM module, read-write memory device, read-only memory device (ROM), random access memory (RAM), flash memory, memory storage device, I-O devices, buttons, display, LED, user interface, rechargeable battery, microphone, speaker, wireless transceiver (e.g., RF, WiFi, Bluetooth, IoT), RF electronic circuits, audio CODEC, cellular antenna, GPS antenna, WiFi antenna, Blue Tooth antenna, IoT antenna, vibrating motor (output), preferably in combination, to function fully as a wireless mobile cellular communication unit. The device can perform one or more executable codes, algorithms, methods, and/or software instructions for automated voice recognition-response, natural language understanding-processing, and wireless mobile cellular communication. One or more components (e.g., buttons, display, user interface, speakers, microphone) are configured to be accessible to a user, on the external surface of a unit/housing.

In another aspect of the present disclosure, an interactive pill dispensing apparatus incorporates, inside or user accessible, one or more storage receptacles for storing one or more pills, pill inventory sensor, environmental sensor, sensor detection/signal processor, analog digital convertor, an actuator for dispensing one or more pills from the storage receptacle, a receptacle for receiving a dispensed pill, user identification component (e.g. fingerprint detector), a charge-coupled device (CCD) camera, data reader (e.g., barcode reader/scanner), RFID reader. In an alternative embodiment, the apparatus may be configured to receive, by insertion into one or more compartment, at least one self-contained pill storage unit (e.g. cartridge, bottle, etc.). The self-contained pill storage unit can possess a data storage component (e.g., linear barcode, matrix barcode, RFID). The inserted unit is controllable to allow the dispensing apparatus to access pills and dispense them under automated instructions. These additional components may function in combination with said computing components (e.g. processors, memory devices, etc.) to perform one or more executable codes, algorithms, methods, and/or software instructions for motor actuation, user identification, image capture, image processing, sensor detection, sensor signal processing, or the like, preferably to function as an advanced pill dispensing apparatus. The user may access (i.e., accessible) or interact with device functions through one or more button, fingerprint detector, camera, touch screen, voice-controlled speech user interface, or smart phone app.

According to aspects of the present disclosure, the interactive pill dispensing apparatus can function in combination with an application software platform accessible to multiple clients (users) executable on one or more remote servers, to preferably establish the communication ecosystem. The ecosystem enables communication and social networking for a user (patient), family, caregivers, healthcare providers, and service providers. Furthermore, the device may function in combination with one or more remote servers, cloud control services, to perform natural language or speech-based interactions with the user, preferably through a voice-controlled speech user interface.

In one embodiment, the interactive pill dispensing apparatus can be configured to function in combination with one or more external devices or smart appliances possessing AI digital assistant capabilities. The user may interact with the AI digital assistant, through a voice-controlled speech user interface, to access or control the pill dispensing apparatus. The AI digital assistant can include, for example, Amazon Alexa®, Google Assistant™, Apple Siri, Microsoft Cortana, Samsung's Bixby, or the like. The interactive pill dispenser and digital assistant capable devices may be paired and/or programmed using a smart phone app or through cloud services, APIs, associated with said digital assistant capable devices. Exemplary digital assistant capable devices include but are not limited to Echo®, Dot®, Show® from Amazon and Google Home®. In another embodiment, one or more clients can communicate with the interactive pill dispensing apparatus using a remote smart appliance, possessing AI digital assistant capabilities, smart phone, smart phone apps, lap top, desk top, or the like.

According to another aspect of the present disclosure, the voice-controlled speech user interface of the advanced pill dispenser detects or monitors audio input/output and interacts with a user to determine a user intent based on natural language understanding of the user's speech. The voice-controlled speech user interface is configured to capture user utterances and provide them to the control service. The combination of the speech interface device and one or more applications executed by the control service serves as a relational agent or AI digital assistant. The relational agent provides conversational interactions, utilizing automated voice recognition-response, natural language processing, predictive algorithms, and the like, to interact and fulfill user requests. These interactions between the relational agent and the user may include but are not limited to scheduling dosing time, alerting user, via voice or audible sound, that a pill has been dispensed, reminding user to take medication, answering medication related questions, querying user status, contacting 911 emergency services, scheduling transportation services (e.g. Uber®, Circulation™), recording and sending messages (e.g., text, voice, video), scheduling doctor's appointments, contacting physicians to obtain refill prescriptions, contacting pharmacists/PMBs to refill prescriptions, facilitating video communication, or the like.

According to another aspect of the present disclosure, the interactive pill dispensing apparatus can communicate with a secured HIPAA-compliant remote server. The remote server is accessible through one or more computing devices, including but not limited to, desktop, laptop, tablet, mobile phone, smart appliances (i.e. smart TVs), or the like. The remote server contains a support application software that include a database for storing user (s) information. The application software provides a collaborative working environment to enable a voluntary, active, and collaborative effort between the advanced pill dispenser user, health care team/providers, caregivers, family members, and service providers. The software environment allows for, but is not limited to, daily tracking of the location of the device, monitoring medication adherence, sending-receiving text messages, push notifications, sending-receiving voice messages, sending-receiving videos, streaming instructional videos, scheduling doctor's appointments, patient education information, caregiver education information, feedback to healthcare providers, feedback to pharmacists, feedback to PBMs, or the like. The application software can be used to store skills relating to the self-management and medication adherence and persistence to therapy. The application software may contain functions for predicting or monitoring user/patient behaviors and non-compliance to pharmacologic therapy. The application software may interact with an electronic health or medical record system (e.g., EMR).

According to yet another aspect of the present disclosure, the secured remote server is also accessible using the stand-alone voice-controlled speech user interface device (i.e. AI digital assistant capable device) or, a speech user interface is incorporated into one or more smart appliance, or mobile app, capable of communicating with the same or another remote server, providing cloud-based control service, to perform natural language or speech-based interaction with the user, acting as the relational agent. The relational agent provides conversational interactions, utilizing automated voice recognition-response, natural language learning-processing, and performs various functions to: interact with the user, fulfill user requests, educate, monitor compliance, monitor persistence, provide one or more skills, ask one or more questions, store responses/answers, perform predictive algorithms with user responses, determine health status and well-being, play games, make phone calls, or provide suggestions for corrective actions including instructions for medication management and adherence.

According to another aspect of the present disclosure, the aforementioned skills are developed and accessible through the relational agent. These skills may include but are not limited to specific educational topics, medication information, prescription insert information, medication nutrition related information (e.g., avoid eating vitamin x while taking warfarin), instructions for taking medication, improving medication adherence, physical rehabilitation, improving cognition, increasing persistence, adverse symptoms management, proprietary developed skills, coping skills, behavioral skills, skills for daily activities, skills for caregivers, or other skills disclosed herein.

A user interacts with the relational agent by providing responses or answers to clinically validated questionnaires or instruments. The questionnaires enable the monitoring of patient behaviors, medication compliance, medication adherence, medication persistence, wellness, symptoms, adverse events monitoring, and the like. The responses or answers provided to the relational agent serve as input to one or more predictive algorithms to calculate a risk stratification profile and trends. Such a profile can provide an assessment for the need for any intervention or behavior modification required by either the user, caregivers, family members, or healthcare team/providers.

Specific embodiments of the present disclosure provide for an interactive pill dispensing apparatus comprising: a housing comprising at least one pill storage receptacle; a pill inventory sensor operably engaged with the at least one pill storage receptacle; an actuator operably engaged with the at least one pill storage unit and being configured to selectively dispense one or more pills; an input/output device configured to receive a user authentication input and a prescription authentication input; and, a control unit being operably engaged with the pill inventory sensor, the actuator, and the input/output device, the control unit comprising at least one processor and at least one non-transitory computer-readable medium having one or more instructions stored thereon to cause the processor to perform one or more actions, the one or more actions comprising: authenticating a user based on the user authentication input; authenticating a prescription based on the prescription authentication input; receiving an input from the pill inventory sensor; determining a pill inventory according to the input from the pill inventor sensor; and, engaging the actuator to dispense one or more pills from the at least one pill storage unit.

Further specific embodiments of the present disclosure provide for an interactive pill dispensing system comprising a housing comprising at least one pill storage receptacle; a pill inventory sensor operably engaged with the at least one pill storage receptacle; an actuator operably engaged with the at least one pill storage unit and being configured to selectively dispense one or more pills; an input/output device configured to receive a user authentication input and a prescription authentication input; a control unit being operably engaged with the pill inventory sensor, the actuator, and the input/output device, the control unit comprising a wireless communication chipset, at least one processor and at least one non-transitory computer-readable medium having one or more instructions stored thereon to cause the processor to perform one or more actions, the one or more actions comprising authenticating a user based on the user authentication input, engaging the actuator to dispense one or more pills from the at least one pill storage unit according to one or more dispensing rules, and storing dispensing data in the at least one non-transitory computer-readable medium; and, a mobile electronic device communicably engaged with the control unit via at least one wireless communications protocol, the mobile electronic device being configured to receive and display dispensing data from the control unit.

Still further specific embodiments of the present disclosure provide for an interactive pill dispensing system comprising: a housing comprising at least one pill storage receptacle; a pill inventory sensor operably engaged with the at least one pill storage receptacle; an actuator operably engaged with the at least one pill storage unit and being configured to selectively dispense one or more pills; an input/output device configured to receive a user authentication input and a prescription authentication input; a control unit being operably engaged with the pill inventory sensor, the actuator, and the input/output device, the control unit comprising a wireless communication chipset, at least one processor and at least one non-transitory computer-readable medium having one or more instructions stored thereon to cause the processor to perform one or more actions, the one or more actions comprising authenticating a user based on the user authentication input, engaging the actuator to dispense one or more pills from the at least one pill storage unit according to one or more dispensing rules, and storing dispensing data in the at least one non-transitory computer-readable medium; and, a remote application server communicably engaged with the control unit via at least one wireless communications network, the remote application server comprising an application database and an application software, the application software being configured to establish one or more secure user associations and one or more secure device associations with the control unit.

In summary, the system comprises an advanced pill dispensing apparatus, functioning as an integral component of an ecosystem for medication management and patient adherence. It utilizes computing, mobile communication, networking, and Artificial Intelligence (AI) to enable a user to be connected to a support system of care givers, healthcare providers, and service providers. The advanced capabilities and functions can be employed to decrease patient non-compliance in an optimal manner.

### BRIEF DESCRIPTIONS OF THE DRAWINGS

The features and components of the following figures are illustrated to emphasize the general principles of the present disclosure. Corresponding features and components throughout the figures can be designated by matching reference characters for the sake of consistency and clarity.

FIG. 1 is a schematic diagram illustrating the components of a pill dispensing apparatus constructed according to an aspect of the present disclosure.

FIG. 2 is a block diagram illustrating the elements of the interactive pill dispenser ecosystem.

FIG. 3 is a screen-shot illustrating information that users can generate using an application software platform of an implementation of the present disclosure.

FIG. 4 illustrates the interactive pill dispensing ecosystem incorporating a multimedia device.

FIG. 5 illustrates a figurative relational agent comprising the voice-controlled speech user interface of the pill dispensing apparatus.

FIG. 6 illustrates the AI digital assistant capable device configured to communicate with the interactive pill dispensing apparatus.

### DETAILED DESCRIPTION

The present disclosure can be understood more readily by reference to the following detailed description, examples, drawings, and claims, and their previous and following description. However, before the present devices, systems, and/or methods are disclosed and described, it is to be understood that this disclosure is not limited to the specific devices, systems, and/or methods disclosed unless otherwise specified, as such can, of course, vary. It is also to be understood that the terminology used herein is for the purpose of describing particular aspects only and is not intended to be limiting.

The following description is provided as an enabling teaching of the present devices, systems, and/or methods in their best, currently known aspect. To this end, those skilled in the relevant art will recognize and appreciate that many changes can be made to the various aspects described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.” Like numbers refer to like elements throughout.

The terminology used herein is for describing particular embodiments only and is not intended to be limiting. As used herein, the singular forms “a,” “an,” and “the” are intended to include the plural forms as well, unless the context clearly indicates otherwise. It will be further understood that the terms “comprises,” “comprising,” “includes,” and/or “including,”, and variants thereof, when used herein, specify the presence of stated features, steps, operations, elements, and/or components, but do not preclude the presence or addition of one or more other features, steps, operations, elements, components, and/or groups thereof.

It will be understood that when an element is referred to as being “coupled,” “connected,” or “responsive” to another element, it can be directly coupled, connected, or responsive to the other element, or intervening elements may also be present. In contrast, when an element is referred to as being “directly coupled,” “directly connected,” or “directly responsive” to another element, there are no intervening elements present. As used herein, the term “and/or” includes any and all combinations of one or more of the associated listed items.

Spatially relative terms, such as “above,” “below,” “upper,” “lower,” “top, “bottom,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” other elements or features would then be oriented “above” the other elements or features. Thus, the term “below” can encompass both an orientation of above and below. The device may be otherwise oriented (rotated 90 degrees or at other orientations) and the spatially relative descriptors used herein interpreted accordingly. Well-known functions or constructions may not be described in detail for brevity and/or clarity.

It will be understood that, although the terms “first,” “second,” etc. may be used herein to describe various elements, these elements should not be limited by these terms. These terms are only used to distinguish one element from another. Thus, a first element could be termed a second element without departing from the teachings of the present embodiments.

Unless otherwise defined, all terms (including technical and scientific terms) used herein have the same meaning as commonly understood by one of ordinary skill in the art to which these embodiments belong. It will be further understood that terms, such as those defined in commonly-used dictionaries, should be interpreted as having a meaning that is consistent with their meaning in the context of the relevant art and will not be interpreted in an idealized or overly-formal sense unless expressly so defined herein.

Several alternative embodiments of the advanced pill dispensing system, apparatus, and methods for medication management and adherence are described herein. Such a system and apparatus may increase user compliance with medication therapy. A principle of the present disclosure is the use of an ecosystem to facilitate a high level of interaction between a user of the pill dispenser with one or more caregiver, family member, healthcare team/provider (e.g., physician), or service provider (e.g., pharmacist). The system leverages a low cognitive demand, voice-controlled empathetic relational agent or AI digital assistant for device access, device operation, education, social support, social contact, support of daily living activities, safety, support for caregivers, feedback/communication for and between healthcare team/providers, and the like. The platform also aids the user to overcome barriers to medication adherence and increase compliance for health and well-being. In one embodiment, the platform or system comprises a combination of at least one of the following components: a pill dispenser; cellular communication module; computing module; communication network; remote server; cloud server; cloud application software. The cloud server and service are commonly referred to as “on-demand computing”, “software as a service (SaaS)”, “platform computing”, “network-accessible platform”, “cloud services”, “data centers,” and the like. The cloud server is preferably a secured HIPAA-compliant remote server. In an alternative embodiment, the system comprises a combination of at least one voice-controlled speech user interface; AI digital assistant capable device; computing device; communication network; remote server; cloud server; cloud application software. These components are configured to function together to enable a user to interact with a resulting relational agent or AI digital assistant. In addition, an application software, accessible by the user and others, using one or more remote computing devices, provides a social, caregiver, healthcare provider, service provider, establishes a network support system for the user.

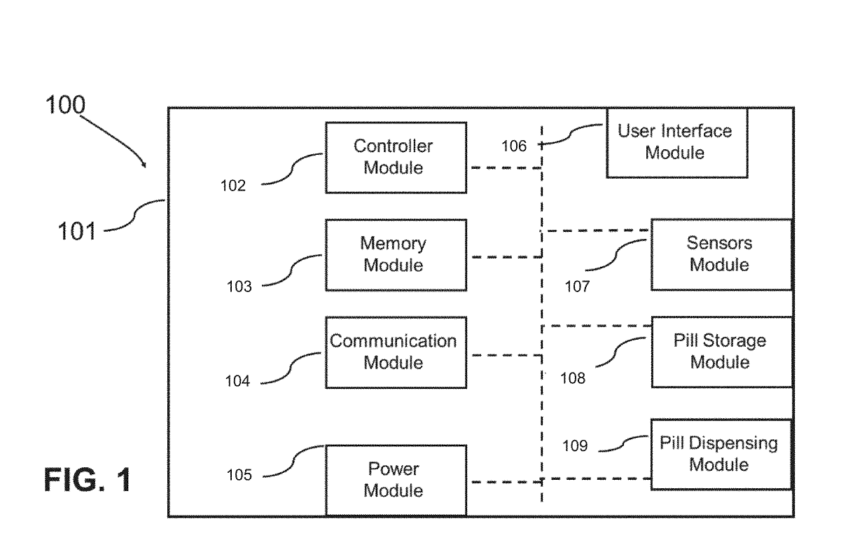

Referring to FIG. 1, schematic diagram 100 illustrates the components that may be configured and/or incorporated, without limitation to, into an interactive pill dispensing apparatus 101 according to an aspect of the present disclosure. The interactive pill dispensing apparatus 101 incorporates, within a self-contained portable unit, one or more modules. Controller Module 102 can comprise one or more microprocessor, microcontroller, computing device, or the like, configured and connected (shown as dotted lines) to communicate and/or to control one or more modules incorporated within the pill dispensing apparatus 101. Controller module 102 can be connected to communicate, access, and/or control the operation and functions of Memory Module 103, which may be comprised of one or more read-write memory device, read-only memory device (ROM), random access memory (RAM), flash memory, memory storage device, or the like. One or more said memory storage device may be configured with instruction code, executable code, binary code, nano-code, microcode, software, or the like, for the operation and function of one or more module of the said pill dispenser. Memory Module 103 can also store executable instructions, for the operation and function of the interactive pill dispensing apparatus 101. Controller Module 102 can be connected to communicate, access, and/or control the operation and functions of Communication Module 104 which may be comprised of one or more wireless transceiver (e.g., RF, WiFi, Bluetooth, IoT), RF electronic circuits, RF transceiver, wireless transceiver, micro GSM/GPRS chipset, micro SIM component, cellular antenna, GPS antenna, WiFi antenna, Bluetooth® antenna, IoT antenna, to function fully as a wireless mobile cellular communication unit, providing wireless communication capabilities. Controller Module 102 can be connected to communicate, access, and/or control the operation and functions of Power Module 105 which may comprise at least one battery or rechargeable battery, rechargeable in conjunction with external power source through direct contact or non-contact (e.g. wireless induction). Power Module 105 can incorporate a fuel/power gauge, to monitor energy consumption and life of the battery, a switched-mode power supply unit, or and low dropout regulator (LDO), to convert electrical power efficiently, eliminate switching noise, and provide simplicity in design. Controller Module 102 can be connected to communicate, access, and/or control the operation and functions of User Interface Module 106 which may comprise one or more button, display, LED, LED display, E-Ink display, touch screen, microphone, speaker, audio CODEC, vibrating motor, or the like. Similarly, Controller Module 102 can be connected to communicate, access, and/or control the operation and functions of the Sensors Module 107 which may comprise one or more sensors including temperature, humidity, pill counter, mass balance, piezo-electric, MEMS, infrared, CCD camera (function, but not limited to, capture user compliance), RFID, optical reader, or the like. Controller Module 102 can be connected to communicate, access, and/or control the operation and functions of the Pill Storage Module 108 which may comprise one or more receptacle, means for receiving an external pill storage unit (e.g., cartridge, bottle, pill pack, etc.), means for detecting insertion/presence of the said pill storage unit, means for ejecting spent pill storage unit, or related functions. Controller Module 102 can be connected to communicate, access, and/or control the operation and functions of the Pill Dispensing Module 109 which may comprise one or more actuators or means to access Pill Storage Module 108 to retrieve and dispense one or more pills, the dispensing receptacle accessible externally to the user, the actuators to open access to or close said dispensing receptacle. It is understood that the modules and components can be integrated within a variety of form factors. For illustration purposes, the physical form factors may include, but are not limited to, an apparatus of the present disclosure, a self-contained, compact box, a miniature device, a portable unit, a wearable dispenser, or the like.

Wireless communication in the ecosystem of the interactive pill dispensing apparatus 101 can include a cellular communication that uses at least one of long-term evolution (LTE), LTE advanced (LTE-A), code division multiple access (CDMA), wideband CDMA (WCDMA), universal mobile telecommunication system (UMTS), wireless broadband (WiBro), and global system for mobile communication (GSM). The wireless communication may include at least one of wireless fidelity (WIFI), Bluetooth™, Bluetooth® low energy (BLE), ZigBee™, near field communication (NFC), magnetic secure transmission, radio frequency (RF), Internet of Things (IoT), or body area network (BAN). The wireless communication may include a global positioning system (GPS), global navigation satellite system (GNSS), Beidou navigation satellite system (Beidou), Galileo, and the European global satellite-based navigation system. Herein, “GPS” may be interchangeably referred to as “GNSS.” Additional bands and equivalent terminologies include Third Generation (3G), Fourth Generation (4G), Fifth Generation (5G), future generations, and the like.

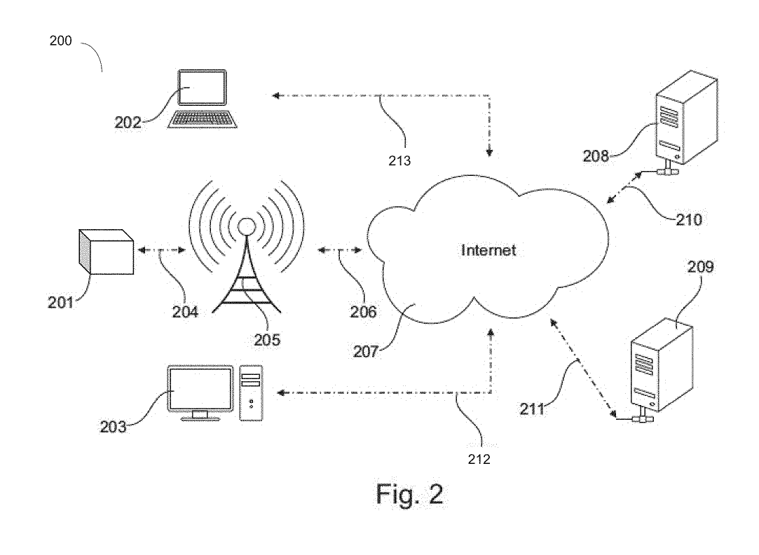

Various implementations of the present disclosure can further utilize an application software platform to comprise the ecosystem for communication and social networking between one or more pill dispenser user, family, caregiver, healthcare provider, and/or service provider (e.g. pharmacist, PBM). FIG. 2 is a block diagram of the elements of the aforementioned ecosystem, identified as 200. One or more user can access the ecosystem 200 using a portable computing device 202 or stationary computing device 203. Computing device 202 may be a laptop used by a family member or caregiver. Stationary computing device 203 may reside at the facility of a healthcare provider (e.g., physician's office) or service provider (e.g., pharmacist). An interactive pill dispensing device 201, corresponding to the interactive pill dispensing apparatus 101 of FIG. 1, communicates with the ecosystem 200 via communication means 204 to one or more cellular communication networks, such as at 205, which can connect device 201 via communication means 206 to the Internet 207. Device 201, 202, and 203 can access one or more remote servers 208, 209 via the Internet 207 through communication means 210 and 211 depending on the server. Device 202 and 203 can access one or more servers through communication means 212 and 213. The application software platform can be stored in one or more of the servers 208, 209. The software environment allows for, but is not limited to, daily tracking of user location, monitoring of pill dispenser user medication adherence, monitoring of user medication persistence, storing and tracking health data (i.e. blood pressure, glucose, cholesterol, etc.), sending-receiving text messages, push notification, sending-receiving voice messages, sending-receiving videos, streaming instructional videos, scheduling doctor's appointments, pill user education information, caregiver education information, feedback to healthcare providers, feedback to pharmacists, and the like. The application software can be used to store skills relating to medication management. The application software may contain functions for predicting pill user behaviors, predicting non-compliance to medication therapy, functions for suggesting corrective actions, functions to improve compliance, games to improve compliance, or the like. The application software may interact with an electronic health or medical record system (e.g., EMR).

In a preferred embodiment, the device 201 enables communication with one or more remote servers, for example server 208, capable of providing cloud-based control service, to perform natural language or speech-based interaction with the user. Device 201 detects audio inputs/listens and interacts with a user to determine a user intent based on natural language understanding of the user's speech. The device 201 is configured to capture user utterances and provide them to the control service located on server 208. The control service performs speech recognition-response and natural language understanding-processing on the utterances to determine intents expressed by the utterances. In response to an identified intent, the controlled service causes a corresponding action to be performed. An action may be performed at the control service or by instructing the device 201 to perform a function. The combination of the device 201 and control service located on remote server 208 serves as a relational agent or AI digital assistant. The relational agent or AI digital assistant provides conversational interactions, utilizing automated voice recognition-response, natural language processing, predictive algorithms, and the like, to: perform functions, interact with the user, fulfill user requests, educate the user, monitor user compliance, determine user health status, user well-being, suggest corrective user actions-behaviors, and the like. The relational agent may fulfill specific requests including calling a family member, a healthcare provider, a pharmacist, or arranging a ride (e.g., Uber®, Circulation™) for the user. In an emergency, for example, a fall event, the user may request the relational agent or AI digital assistant to contact an emergency service. Ultimately the device 201 enables the user to access and interact with the said relational agent or AI digital assistant to provide user education, instructions for taking medication, informing a patient that a pill has been taken, support, social contact support, support of daily activities, user safety, record of diary entry/contents, support for caregivers, feedback/communication for healthcare team/providers, pharmacist, or the like, for medication management. The information generated from the interaction of the user and others can be captured and stored in a remote server, for example remote server 209. This information may be incorporated into the application software making it accessible to multi-users (e.g. healthcare team, caregiver, pharmacist, etc.) of the ecosystem 200.

In an alternative embodiment, the function of the relational agent or AI digital assistant can be accessed through a mobile app and implemented through the ecosystem 200 illustrated in FIG. 2. Such mobile app provides access to a remote server, for example remote server 208 of FIG. 2, capable of providing cloud-based control service, to perform natural language or speech-based interaction with the user. The mobile app contained in a mobile cellular unit (e.g., mobile phone) monitors and captures voice commands and/or utterances and transmits them through the said communication means to the control service located on server 208. The control service performs speech recognition-response and natural language understanding-processing on the utterances to determine intents expressed by the utterances. In response to an identified intent, the control service causes a corresponding action to be performed. An action may be performed at the control service or by responding to the user through the mobile app. The control service located on remote server 208 serve as a relational agent or AI digital assistant. The relational agent or AI digital assistant provides conversational interactions, utilizing automated voice recognition-response, natural language processing, predictive algorithms, and the like, to perform functions, interact with the user, fulfill user requests, educate, monitor compliance, determine health status, well-being, suggest corrective actions-behaviors, and the like. The interactive pill dispensing apparatus (101, 201) enables the user to access and interact with the relational agent or AI digital assistant for medication management. The information generated from the interaction of the user and the relational agent can be captured and stored in a remote server, for example remote server 209. This information may be incorporated into the application software as described in FIG. 2, making it accessible to multi-users of the ecosystem 200.

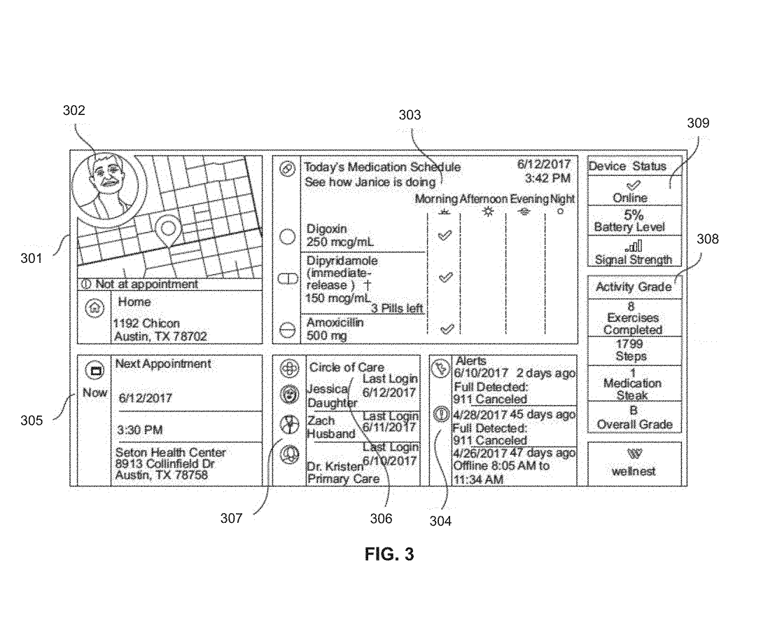

FIG. 3 is a screen-shot 301 that illustrates the type of information that users can generate using the application software platform. Screen-shot 301 provides an example of the information arranged in a specific manner and by no means limits the potential alternative or additional information that can be made available and displayed by the application software. In this example, a picture of a pill dispenser user 302 is presented at the upper left corner. The application may display the current location of the pill dispenser user 302, providing real-time location of the device. This may be useful in the event of misplacement, theft, or prevention of abuse of a controlled substance (e.g. opioids). A Medication Schedule 303 is available for review and contains a list of medications, dosage, and time when taken. This may be useful for caregivers, healthcare providers, or pharmacist in the monitoring of user compliance and refills. An Alerts 304 is also visible that documents fall detection events and 911 emergency connections for the pill dispenser user 302. The pill dispenser user can review the Next Appointment 305 information, for example for returning to a physical therapy session. A Circle of Care 306 has pictures of the people 307 (i.e. family members) interacting with the user 302 in the said ecosystem and log-in information. Lastly, but not least, Device Status 909 provides information on the status of said device.

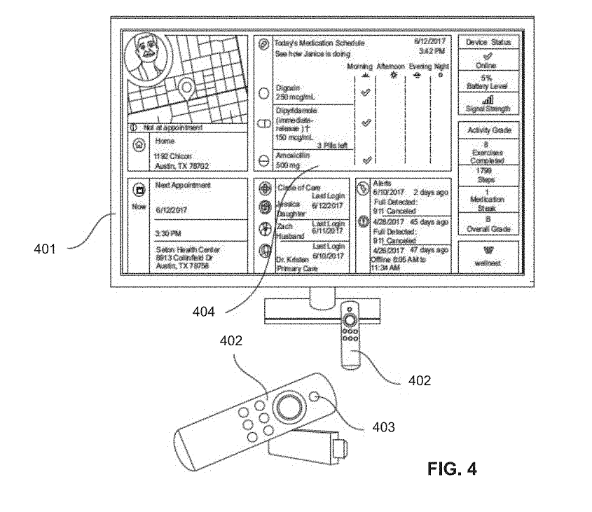

FIG. 4 illustrates the ecosystem incorporating a multimedia device 401 for a pill dispenser user or the like to interact with one or more remote healthcare team/provider, caregiver, or family member through a relational agent or AI digital assistant. One or more user can access the system using a remote-controlled device 402 containing a voice-controlled speech user interface 403. The multimedia device 401 is configured to enable a user to access application software platform depicted by screen-shot 404. The multimedia device 401 may be configured with hardware and software that enables streaming videos to be displayed. Exemplary products include FireTV®, Fire HD8 Tablet, Echo Show®; products available from Amazon.com (Seattle, Wash.), Nucleus (Nucleuslife.com), Triby (Invoxia.com), TCL Xcess, or the like. Streaming videos may include educational contents or materials to improve user (patient) behavior, cognition, knowledge, attitudes, and practices to improve medication adherence and outcomes, or general health outcomes. Preferable materials include contents and tools to increase user knowledge and understanding of medication, for example nutritional influences on medication, or the like.

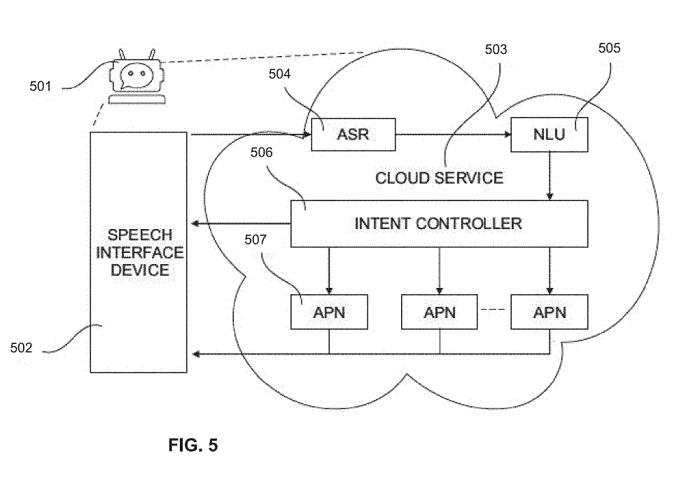

FIG. 5 illustrates a figurative relational agent or AI digital assistant 501 comprising the voice-controlled speech user interface device 502, corresponding to device 101 of FIG. 1, and a cloud-based control service 503. A representative cloud-based control service can be implemented through a SaaS model or the like. Model services include, but are not limited to, Amazon Web Services, Amazon Lex™, Amazon Lambda™, Google Assistant™, Apple Siri®, Microsoft Cortana®, Samsung's Bixby®, or the like. Such a service provides access to one or more remote servers containing hardware and software to operate in conjunction with said voice-controlled speech interface device, said AI digital assistant capable device, app, or the like. Without being bound to a specific configuration, said control service may provide speech services implementing an automated speech recognition (ASR) function 504, a natural language understanding (NLU) function 505, an intent router/controller 506, and one or more applications 507 providing commands back to the voice-controlled speech interface device, AD digital assistant capable device, smart phone app, or the like. The ASR function can recognize human speech in an audio signal transmitted by the voice-controlled speech interface device received from a built-in microphone. The NLU function can determine a user intent based on user speech that is recognized by the ASR components. The speech services may also include speech generation functionality that synthesizes speech audio. The control service may also provide a dialog management component configured to coordinate speech dialogs or interactions with the user in conjunction with the speech services. Speech dialogs may be used to determine the user intents using speech prompts. One or more applications can serve as a command interpreter that determines functions or commands corresponding to intents expressed by user speech. In certain instances, commands may correspond to functions that are to be performed by the voice-controlled speech user interface device and the command interpreter may in those cases provide device commands or instructions to the voice-controlled speech user interface device for implementing such functions. The command interpreter can implement “built-in” capabilities that are used in conjunction with the voice-controlled speech user interface device. The control service may be configured to use a library of installable applications including one or more software applications or skill applications of the present disclosure. The control service may interact with other network-based services (e.g., Amazon Lambda, Google Cloud) to obtain information, access additional database, application, or services on behalf of the user. A dialog management component is configured to coordinate dialogs or interactions with the user based on speech as recognized by the ASR component and/or understood by the NLU component. The control service may also have a text-to-speech component responsive to the dialog management component to generate speech for playback on the voice-controlled speech user interface device. These components may function based on models or rules, which may include acoustic models, specify grammar, lexicons, phrases, responses, and the like created through various training techniques. The dialog management component may utilize dialog models that specify logic for conducting dialogs with users. A dialog comprises an alternating sequence of natural language statements or utterances by the user and system generated speech or textual responses. The dialog models embody logic for creating responses based on received user statements to prompt the user for more detailed information of the intents or to obtain other information from the user. An application selection component or intent router identifies, selects, and/or invokes installed device applications and/or installed server applications in response to user intents identified by the NLU component. In response to a determined user intent, the intent router can identify one of the installed applications capable of servicing the user intent. The application can be called or invoked to satisfy the user intent or to conduct further dialog with the user to further refine the user intent. Each of the installed applications may have an intent specification that defines the serviceable intent. The control service uses the intent specifications to detect user utterances, expressions, or intents that correspond to the applications. An application intent specification may include NLU models for use by the natural language understanding component. In addition, one or more installed applications may contain specified dialog models that create and coordinate speech interactions with the user. The dialog models may be used by the dialog management component in conjunction with the dialog models to create and coordinate dialogs with the user and to determine user intent either before or during operation of the installed applications. The NLU component and the dialog management component may be configured to use the intent specifications of the applications either to conduct dialogs, to identify expressed intents of users, identify and use the intent specifications of installed applications, in conjunction with the NLU models and dialog modes, to determine when a user has expressed an intent that can be serviced by the application, and to conduct one or more dialogs with the user. As an example, in response to a user utterance, the control service may refer to the intent specifications of multiple applications, including both device applications and server applications, to identify a “Pill Dispenser” intent. The service may then invoke the corresponding application. Upon invocation, the application may receive an indication of the determined intent and may conduct or coordinate further dialogs with the user to elicit further intent details. Upon determining sufficient details regarding the user intent, the application may perform its designed functionality in fulfillment of the intent. The voice-controlled speech interface device in combination with one or more functions 504,505,506 and applications 507 provided by the cloud service represents the relational agent or AI digital assistant 501.

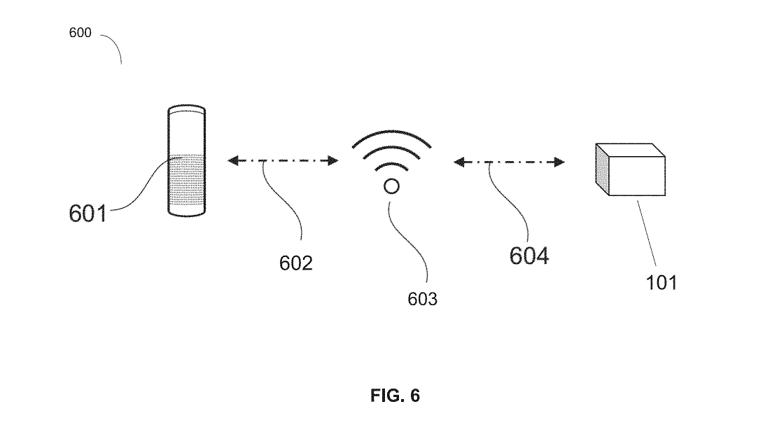

Referring to FIG. 6, illustration 600 depicts device 601, corresponding to device 501 of FIG. 5, which can be configured to communicate with the interactive pill dispensing apparatus 101 of FIG. 1. Device 601 communicates with the interactive pill dispensing apparatus 101 via communication means 602 to one or more WiFi communication network 603 which can connect device 601 via communication means 604 to pill dispenser 101. In an alternative configuration, device 601 may communicate directly with the interactive pill dispensing apparatus 101 via, for example, but not limited to, Bluetooth. The user may interact with the AI digital assistant, through a voice-controlled speech user interface 601, to access or control the interactive pill dispensing apparatus 101. The AI digital assistant may include but is not limited to Amazon Alexa®, Google Assistant™, Apple Siri®, Microsoft Cortana®, Samsung's Bixby®, or the like. The pill dispenser and digital assistant capable devices may be paired and/or programmed using a smart phone app or through cloud services, APIs, associated with said digital assistant capable devices. Exemplary digital assistant capable devices include but are not limited to Echo®, Dot®, Show® from Amazon and Google Home®. In another embodiment, one or more clients can communicate with the said advanced pill dispensing apparatus using a remote smart appliance, possessing AI digital assistant capabilities, smart phone, smart phone apps, lap top, desk top, or the like.

In a preferred embodiment, skills are developed for the relational agent or AI digital assistant 501 of FIG. 5 and stored as accessible applications within the cloud service 503. The skills contain information that enables the relational agent or AI digital assistant to respond to intents by performing an action in response to a natural language user input, information of utterances, spoken phrases that a user can use to invoke an intent, slots or input data required to fulfill an intent, and fulfillment mechanisms for the intent. These application skills may also reside in an alternative remote service, remote database, the Internet, or the like, and yet be accessible to the cloud service 503. These skills may include but are not limited to intents for general topics, weather, news, music, pollen counts, UV conditions, user/patient engagement skills, nutrition, instructions for taking medication, prescription instructions, medication adherence, persistence, or the like. The skills enable the relational agent 501 to respond to intents and fulfill them through the voice-controlled speech interface device. These skills may be developed using application tools from vendors (e.g., Amazon Web Services®, Alexa® Skill Kits, Google, Apple, Samsung) providing cloud control services. The pill dispenser user preferably interacts with relational agent or AI digital assistant 501 using skills that enable a voluntary, active, and collaborative effort between a patient with caregivers, family members, health care team/providers, or pharmacists.

Exemplary skills accessible to a patient may be one or more medication management interventions including self-efficacy skills, self-management skills, medication adherence skills, medication persistence skills, or the like. A relational agent or AI digital assistant can be provided with skills to be able to fulfill one or more intents invoked by a patient, for example: symptoms identification (e.g. medication adverse symptoms, etc.) and management skills (e.g., invoke relational agent or AI digital assistant to call 911). It is a preferred object to utilize the spoken language interface as a natural means of interaction between the users and the system. Users can speak to the AI digital assistant similarly as they would normally speak to a human. It is understood, but not bound by theory, that verbal communication accompanied by the opportunity to engage in meaningful conversations can reinforce, improve, and motivate behavior for simultaneous self-management of medication. The relational agent or AI digital assistant may be used to engage the patient in activities aimed at stimulating social functioning to leverage social support for improving compliance and persistence. These skills may create a user-centered environment for user-centered care. Preferred skills are those, but not limited to, that examine important psychological or social constructs or utilize informative tools for assessing and improving patient compliance. The preferred environment is a collaborative relationship between providers and users, in a shared decision-making and a personal systems approach; both have the potential to improve medication adherence and/or mobility outcomes.

The relational agent and one or more skills may be implemented in the engagement of a patient at an ambulatory setting (i.e. home, physician's office, clinic, etc.). During a session, the relational agent using one or more skills may inform the user about, for example, proper dosing. Skills may include topics of instructions to prevent medication complications. The relational agent may inform the patient about the possible problems that might be encountered with operations of the dispenser. The relational agent may inform the patient that the pill dispenser is low on pills or that pills are about to expire. The relational agent may monitor the pill dispenser and subsequently send a status to a caregiver, a healthcare provider, or a pharmacist. For example, the relational agent may request a refill to be sent to the user's home based on the inventory of pills in the Pill Storage Module 108 of FIG. 1. Similarly, the relational agent may inform a healthcare provider that the patient needs a prescription for refill. The platform herein disclosed allows remote monitoring by family members, a healthcare team/provider (e.g., nurse, clinician, specialist, therapist) on quantity or quality (e.g. mean, standard deviation, frequency, etc.) of medication management, compliance, persistence, by users/patients and intervene as necessary. For example, a family member may access the dispenser remotely using a smart phone to determine compliance with medication administration.

Various implementations of the present disclosure can utilize a means to assess knowledge of specific diseases, physical, mental, neural, or cognitive health conditions, monitor medication adherence, and assess the emotional well-being of pill dispenser user using a standard set of validated questionnaires and/or patient-reported outcomes (PROs) instruments. The responses-answers provided or obtained from these questionnaires and instruments enable the assessment of a user's physical functioning, psychological functioning, and overall health-related QoL. This may be implemented using clinically validated questionnaires conducted by the relational agent or AI digital assistant. Upon a user intent, the relational agent can execute an algorithm or a pathway consisting of a series of questions that proceed in a state-machine manner, based upon yes or no responses, or specific response choices provided to the user. For example, a clinically validated structured multi-item, multidimensional, questionnaire scale may be used to assess knowledge of physical therapy, health conditions, or symptoms, self-efficacy, or the like. The scale is preferably numerical, qualitative or quantitative, and allows for concurrent and predictive validity, with high internal consistency (i.e., high Cronbach' s alpha), high sensitivity and specificity. Questions are asked by the relational agent and responses, which may be in the form of yes/no answers from patients or caregivers, are recorded and processed by one or more skills. Responses may be assigned a numerical value, for example yes=1 and no=0. A high sum of yes in this case provides a measure of non-adherence. One of ordinary skill in the art can appreciate the novelty and usefulness of, among other things, using the disclosed relational agent; a voice-controlled speech recognition and natural language processing combined with the utility of validated clinical questionnaire scales or PROs instruments. The questionnaire scales are constructed and implemented using skills developed through for example using the Alexa® Skills Kit and/or Amazon Lex™. The combination of these modalities may be more conducive to eliciting information, providing feedback, and actively engaging patients for medication management.

Clinically validated scales and PROs instruments may be constructed to measure, assess, or monitor the following, without being limited to: physical well-being, social well-being, emotional well-being, functional well-being, pain, fatigue, nausea, sleep disturbance, distress, shortness of breath, loss of memory, loss of appetite, drowsiness, dry mouth, anxiety, sadness, emesis, numbness, bruising, disease specific-related symptoms, or the like; rated on the basis of their presence and severity. PROs instruments may also be constructed to measure, assess, or monitor medication, medication administration, medication interactions, activity, diet, side effects, informing healthcare team/providers, informing pharmacists, and QoL. It is understood that any clinically validated PROs instruments, modified or unmodified, for medication management may be implemented according to various aspects of the present disclosure. All said questionnaires, PRO instruments, scales and the like can be constructed and implemented using the Alexa® Skills Kit and/or Amazon Lex™ system, or the like. User responses provide objective data about different aspects of education and practice that are taught and retained by the user education. Thus, these instruments, for example, served as a good quality control measure of user compliance. Frequently missed questions may indicate potential areas for improvement in user education, including reinforcement of treatment guidelines as well as recommendations to contact healthcare providers or pharmacists for questions. In addition, the relational agent may assess the need for re-education or suggest areas for improvements to keep patients in compliance with medication therapy.

The said scales may be modifiable with variable number of items and may contain sub-scales with either yes/no answers, or response options, response options assigned to number values, Likert-response options, or Visual Analog Scale (VAS) responses. VAS responses may be displayed via mobile app in the form of text messages employing emojis, digital images, icons, and the like.

The results from one or more questionnaire, scales, and PROs instruments may be obtained and/or combined to monitor and provide support for patient education, social contact, daily activities, user safety, support for caregivers, and feedback communication for healthcare providers and pharmacists in medication management. Questionnaire, scales, and PROs instruments may be directed to either caregivers or patients. User responses on the questionnaires are sent to the application software platform. The answers provided to the relational agent serve as input to one or more indices, predictive algorithms, statistical analyses, or the like, to calculate a risk stratification profile and trends. Such a profile can provide an assessment for the need of any intervention (i.e. corrective action) required by either the user, healthcare team/providers, caregivers, pharmacists, or family members. Trends in these symptoms can be recorded and displayed in a graphical format within the application software.

In summary, the disclosed device and the system serve to augment medication management and provide a caregiver, healthcare provider(s), pharmacists, and PBMs social network support system for the user. The system incorporates a voice-controlled empathetic relational agent or AI digital assistant as a simple, user-friendly, natural, user-interface for operating the advanced pill dispenser, user education, general support, social contact support, support with daily living activities, safety, support for caregivers, feedback for healthcare providers, pharmacists, and the like, for medication management. For patients, the system supports their needs regarding, without being limited to, medication adherence, persistence, symptoms management, cognitive aid, cognitive stimulation, coping, emotional support, social support, and educational information. For caregivers, the system supports their needs regarding, without being limited to, monitoring patient compliance and wellbeing. For healthcare team/providers, the system supports their needs regarding, without being limited to, patient behavior, profile, medication adherence, routine adherence, adherence to medication therapy, user/patient health status, and sharing of patient information across multiple healthcare settings (e.g., PCPs, specialists, pharmacists, etc.). The system establishes an ecosystem that is patient-centered, comprehensive, coordinated, accessible (24/7), and enables healthcare team/providers to enhance quality improvement, ensuring that users/patients and families make informed decisions in the management of medication therapy.

### EXAMPLE 1

This example is intended to serve as a demonstration of the possible voice interactions between a relational agent and a patient. The relational agent uses a control service (Amazon Lex™) available from Amazon.com (Seattle, Wash.). Access to skills requires the use of a device wake word (“Alexa”) as well as an invocation phrase (“Pill Dispenser”) for skills specifically developed for the device that embodies one or more components disclosed herein. The following highlights one or more contemplated implementations:

| Feature | Sample Phrases |
|---|---|
| Checking | “Alexa, ask Pill Dispenser if I have any messages” |
| Messages | “Alexa, tell Pill Dispenser to check my messages” |
| Appointment | “Alexa, ask Pill Dispenser when my next appointment |
| Schedule | is.” “Alexa, ask Pill Dispenser about my appointment |
| | schedule.” |
| Fire TV | “Alexa, ask Pill Dispenser what is new on Fire TV about |
| Video Content | Lipitor” “Alexa, ask Wellwalk if there is anything new |
| | on Fire TV about my number of steps and Performance.” |
| Alert | Pill Dispenser: “It's time to take your medication” |
| | Pill Dispenser: “You have already taken that medication” |
| Emergency | “Alexa, tell Pill Dispenser to call 911. I have fallen and |
| Assistance | can't get up” “Alexa, ask Pill Dispenser to call an |
| | ambulance” |
| Contact Family | “Alexa, tell Pill Dispenser to call Hannah” |
| Pharmacist | “Alexa, tell Pill Dispenser to call my pharmacist” |
| Medication | “Alexa, tell Pill Dispenser I'd like to ask |
| Adherence | how I am doing with my medication” |
| Measure | Alexa: “I would like to ask you several questions. |
| | Would you like to proceed?” |
| | Alexa: “Do you remember what you should do when |
| | realizing that you have forgotten to take your medicine?” |
| | Pill Dispenser: “Please look at the camera while |
| | taking your medication so I can take a picture” |

Many different embodiments have been disclosed regarding the above descriptions and the drawings. It will be understood that it would be unduly repetitious to literally describe and illustrate every combination and sub-combination of these embodiments. Accordingly, the present specification, including the drawings, shall be construed to constitute a complete written description of all combinations and sub-combinations of the embodiments described herein, and of the manner and process of making and using them, and shall support claims to any such combination or sub-combination. In the drawings and specification, there have been disclosed various embodiments and, although specific terms are employed, they are used in a generic and descriptive sense only and not for purposes of limitation. Therefore, it will be apparent to those skilled in the art that various modifications and variations can be made to the disclosed embodiments without departing from the scope or spirit of the present disclosure. All such modifications and variations are intended to be included herein within the scope of the present disclosure, and all possible claims to individual aspects or combinations of elements or steps are intended to be supported by the present disclosure.

What is claimed is:

1. An interactive pill dispensing apparatus comprising:
a housing comprising at least one pill storage unit;
a pill inventory sensor operably engaged with the at least one pill storage unit;
an actuator operably engaged with the at least one pill storage unit and being configured to selectively dispense one or more pills;
an input/output device configured to receive a user authentication input and a prescription authentication input; and,
a control unit being operably engaged with the pill inventory sensor, the actuator, and the input/output device, the control unit comprising at least one processor and at least one non-transitory computer-readable medium having one or more instructions stored thereon to cause the processor to perform one or more actions, the one or more actions comprising:
authenticating a user based on the user authentication input;
authenticating a prescription based on the prescription authentication input;
receiving an input from the pill inventory sensor;
determining a pill inventory according to the input from the pill inventory sensor; and,
engaging the actuator to dispense one or more pills from the at least one pill storage unit.

2. The interactive pill dispensing apparatus of claim 1 further comprising an optical data reader operably engaged with the control unit.

3. The interactive pill dispensing apparatus of claim 1 wherein the at least one pill storage unit is configured as a removable pill storage cartridge.

4. The interactive pill dispensing apparatus of claim 1 wherein the input/output device further comprises a biometric scanner.

5. The interactive pill dispensing apparatus of claim 1 wherein the input/output device further comprises an RFID reader.

6. The interactive pill dispensing apparatus of claim 1 wherein the control unit further comprises a wireless communication chipset.

7. The interactive pill dispensing apparatus of claim 1 further comprising a charge-coupled device camera operably engaged with the control unit.

8. The interactive pill dispensing apparatus of claim 1 wherein the input/output device further comprises at least one microphone.

9. An interactive pill dispensing system comprising:
a housing comprising at least one pill storage unit;
a pill inventory sensor operably engaged with the at least one pill storage unit;
an actuator operably engaged with the at least one pill storage unit and being configured to selectively dispense one or more pills;
an input/output device configured to receive a user authentication input and a prescription authentication input;
a control unit being operably engaged with the pill inventory sensor, the actuator, and the input/output device, the control unit comprising a wireless communication chipset, at least one processor and at least one non-transitory computer-readable medium having one or more instructions stored thereon to cause the processor to perform one or more actions, the one or more actions comprising authenticating a user based on the user authentication input, engaging the actuator to dispense one or more pills from the at least one pill storage unit according to one or more dispensing rules, and storing dispensing data in the at least one non-transitory computer-readable medium; and,
a mobile electronic device communicably engaged with the control unit via at least one wireless communications protocol, the mobile electronic device being configured to receive and display the dispensing data from the control unit.

10. The interactive pill dispensing system of claim 9 wherein the mobile electronic device is configured to receive a voice input from a user and communicate a command prompt to the control unit according to the voice input.

11. The interactive pill dispensing system of claim 9 wherein the mobile electronic device is a smart phone.

12. The interactive pill dispensing system of claim 11 further comprising a mobile software application executing on the smart phone, the mobile software application being configured to define one or more medication delivery controls and monitor one or more medication adherence parameters.

13. The interactive pill dispensing system of claim 9 wherein the one or more actions further comprise authenticating a prescription based on the prescription authentication input and determining a pill inventory according to an input from the pill inventory sensor.

14. The interactive pill dispensing system of claim 12 wherein the mobile software application is further configured to cause the smart phone to deliver one or more medication adherence prompts to the user.

15. An interactive pill dispensing system comprising:
a housing comprising at least one pill storage unit;
a pill inventory sensor operably engaged with the at least one pill storage unit;
an actuator operably engaged with the at least one pill storage unit and being configured to selectively dispense one or more pills;
an input/output device configured to receive a user authentication input and a prescription authentication input;
a control unit being operably engaged with the pill inventory sensor, the actuator, and the input/output device, the control unit comprising a wireless communication chipset, at least one processor and at least one non-transitory computer-readable medium having one or more instructions stored thereon to cause the processor to perform one or more actions, the one or more actions comprising authenticating a user based on the user authentication input, engaging the actuator to dispense one or more pills from the at least one pill storage unit according to one or more dispensing rules, and storing dispensing data in the at least one non-transitory computer-readable medium; and,
a remote application server communicably engaged with the control unit via at least one wireless communications network, the remote application server comprising an application database and an application software, the application software being configured to establish one or more secure user associations and one or more secure device associations with the control unit.

16. The interactive pill dispensing system of claim 15 wherein the application software is further configured to receive the dispensing data from the control unit and execute one or more medication adherence routines in response to the dispensing data.

17. The interactive pill dispensing system of claim 16 further comprising one or more mobile electronic devices communicably engaged with the remote application server to receive medication adherence data from the remote application server.

18. The interactive pill dispensing system of claim 15 further comprising an electronic health or medical record server communicably engaged with the remote application server.

19. The interactive pill dispensing system of claim 15 wherein the input/output device further comprises a voice-controlled speech user interface.

20. The interactive pill dispensing system of claim 19 wherein the remote application server further comprises a natural-language processing function.
